(19) United States
(12) Patent Application Publication
(10) Pub. No.: US 2012/0226343 A1
(43) Pub. Date: Sep. 6, 2012

(54) STENT DELIVERY SYSTEM

(75) Inventors: Justin Vo, San Jose, CA (US); Michael Spencer, Danville, CA (US); Sargon Bourang, San Jose, CA (US); William S. Henry, Oakland, CA (US); Hong Doan, Milpitas, CA (US); Hanh Doan, Milpitas, CA (US)

(73) Assignees: STRYKER NV OPERATIONS LIMITED, Dublin (IE); STRYKER CORPORATION, Kalamazoo, MI (US)

(21) Appl. No.: 13/411,340

(22) Filed: Mar. 2, 2012

Related U.S. Application Data
(60) Provisional application No. 61/449,294, filed on Mar. 4, 2011.

(51) Int. Cl. A61F 2/84

(52) U.S. Cl. 623/1.12

(57) ABSTRACT

A stent delivery system includes a delivery member, a frictional interfacing member disposed on a distal region of the delivery member, the frictional interfacing member comprising a plurality of perfusion channels, a self-expanding stent disposed over the respective frictional interfacing member and delivery member in a radially contracted configuration, and a sheath defining disposed over the respective self-expanding stent, frictional interfacing member, and delivery member, wherein the frictional interfacing member resists axial and/or rotational movement of the stent relative to the delivery member while the stent is in its radially contracted configuration, and wherein the perfusion channels permit fluid to flow from an interior region of the sheath proximal of the frictional interfacing member to an interior region of the sheath distal of the frictional interfacing member.

### RELATED APPLICATION DATA

The present application claims the benefit under 35 U.S.C. §119 to U.S. Provisional Application No. 61/449,294, filed Mar. 4, 2011, the contents of which are incorporated herein by reference as though set forth in full.

### FIELD

The present disclosure relates generally to medical devices and intravascular medical procedures and, more particularly, to devices and methods for delivering a stent to a target site in a blood or other body vessel.

### BACKGROUND

The use of intravascular medical devices has become an effective method for treating many types of vascular disease. In general, a suitable intravascular device is inserted into the vascular system of the patient and navigated through the vasculature to a desired target site. Using this method, virtually any target site in the patient's vascular system may be accessed, including the coronary, cerebral, and peripheral vasculature.

Medical devices such as stents, stent grafts, and vena cava filters are often utilized in combination with a delivery device for placement at a desired location within the body. A medical prosthesis, such as a stent for example, may be loaded onto a stent delivery device and then introduced into the lumen of a body vessel in a configuration having a reduced diameter. Once delivered to a target location within the body, the stent may then be expanded to an enlarged configuration within the vessel to support and reinforce the vessel wall while maintaining the vessel in an open, unobstructed condition. The stent may be configured to be self-expanding, expanded by an internal radial force such as a balloon, or a combination of self-expanding and balloon expandable.

A number of different stent delivery devices, assemblies, and methods are known, each having certain advantages and disadvantages. However, there is an ongoing need to provide alternative stent delivery devices, assemblies, and methods. In particular, there is an ongoing need to provide alternative stent delivery devices that facilitate re-sheathing and repositioning of a stent during the delivery procedure, and methods of making and using such delivery devices and/or assemblies.

### SUMMARY

In one embodiment, a stent delivery system includes a delivery member, a frictional interfacing member disposed on a distal region of the delivery member, the frictional interfacing member comprising a plurality of perfusion channels, a self-expanding stent disposed over the respective frictional interfacing member and delivery member in a radially contracted configuration, and a sheath defining disposed over the respective self-expanding stent, frictional interfacing member, and delivery member, wherein the frictional interfacing member preferably includes a relative high friction outer surface that resists axial and/or rotational movement of the stent relative to the delivery member while the stent is in its radially contracted configuration, and wherein the perfusion channels permit fluid to flow from an interior region of the sheath proximal of the frictional interfacing member to an interior region of the sheath distal of the frictional interfacing member.

The system may optionally further comprise respective proximal and distal bumpers attached to the delivery member and configured to limit respective proximal and distal axial movement of the stent relative to the delivery member while the stent is constrained within the sheath lumen.

At least some of the perfusion channels may be formed in the outer surface of the frictional interfacing member. Alternatively or additionally, the frictional interfacing member has an annular body including a high friction inner surface frictionally secured to the delivery member, wherein at least some of the perfusion channels are formed in the inner surface of the frictional interfacing member. Still further additionally or alternatively, at least some of the perfusion channels comprise ports extending longitudinally through the frictional interfacing member from a proximal facing surface of the frictional interfacing member to a distal facing surface of the frictional interfacing member.

In another embodiment, a method of delivering a stent to a target site in a blood vessel includes (a) providing a stent delivery system including a delivery member, a frictional interfacing member disposed on a distal region of the delivery member, a self-expanding stent disposed over the respective frictional interfacing member and delivery member in a radially contracted configuration, and a sheath disposed over the respective self-expanding stent, frictional interfacing member, and delivery member, (b) introducing liquid into an open proximal end of the sheath, such that the fluid migrates through a plurality of perfusion channels formed in the frictional interfacing member to an interior region of the sheath distal of the frictional interfacing member; (c) advancing the distal region of sheath into a blood vessel until the stent is positioned proximate a deployment site in the vessel, wherein the frictional interfacing member inhibits rotation of the stent relative to the delivery member during said advancing; (d) withdrawing the sheath proximally relative to the stabilized delivery member to thereby unsheathe a distal portion of the stent, wherein the frictional interfacing member inhibits axial movement of the stent relative to the delivery member during said withdrawing, such that a proximal portion of the stent and the frictional interfacing member remain covered by the sheath; (e) determining a position of the unsheathed portion of the stent in the vessel; and (f) if the determined position of the unsheathed portion of the stent is not a desired deployment site in the vessel, advancing the sheath distally relative to the delivery member or withdrawing the delivery member proximally relative to the stabilized sheath to thereby re-sheath the distal portion of the stent. By way of non-limiting example, the act of partially unsheathing a distal portion of the stent may comprise unsheathing a majority, including up to about 80% of the axial length of the stent.

The method may further (optionally) include (g) repositioning the distal region of the sheath and re-sheathed stent within the vessel; (h) repeating acts (d) to (f) until the stent is determined to be at a desired deployment site in the blood vessel; (i) withdrawing the sheath proximally to unsheathe the entire stent and frictional interfacing member; (j) allowing the stent to expand radially and disengage from the frictional interfacing member; and (k) removing the respective sheath, frictional interfacing member, and delivery member from the vessel. In one such embodiment, the method additionally includes monitoring the position of the sheath relative to the frictional interfacing member while withdrawing the sheath proximally over the frictional interfacing member to avoid withdrawing the distal end of the sheath over the frictional interfacing member. By way of non-limiting example, such monitoring may be performed by viewing a radiopaque core of the frictional interfacing member.

Other and further aspects and features of embodiments of the disclosed inventions will become apparent from the ensuing detailed description in view of the accompanying figures.

### BRIEF DESCRIPTION OF THE DRAWINGS

The drawings illustrate the design and utility of embodiments of the disclosed inventions, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments of the disclosed inventions and are not therefore to be considered limiting of its scope.

FIG. 1 is a side view of a stent delivery system constructed according to one embodiment of the disclosed inventions, with a distal region of the system shown in an inset.

FIGS. 2A-2C are respective schematic views of the distal region of a stent delivery system constructed according to one embodiment of the disclosed inventions, illustrating placement of a stent at a target site in a blood vessel.

FIG. 3 is a side view of a delivery wire and a frictional interfacing member constructed according to one embodiment of the disclosed inventions, with select elements shown in shadow for clarity.

FIGS. 4A-4F are detailed longitudinal cross-section views taken along 4-4 in FIG. 2A.

FIGS. 5 and 6 are top views of unrolled stents constructed according to embodiments of the disclosed inventions.

FIG. 7 is a perspective view of a frictional interfacing member constructed according to one embodiment of the disclosed inventions.

FIG. 8 is a sequence of top views of the distal end of a stent delivery system constructed according to one embodiment of the disclosed inventions, showing the delivery wire is being rotated 360 degrees about its longitudinal axis.

FIG. 9 is a sequence of top views of the distal end of a stent delivery system constructed according to one embodiment of the disclosed inventions, showing the stent being re-sheathed.

### DETAILED DESCRIPTION OF THE ILLUSTRATED EMBODIMENTS

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Various embodiments of the disclosed inventions are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. An aspect or an advantage described in conjunction with a particular embodiment of the disclosed inventions is not necessarily limited to that embodiment and can be practiced in any other embodiments even if not so illustrated.

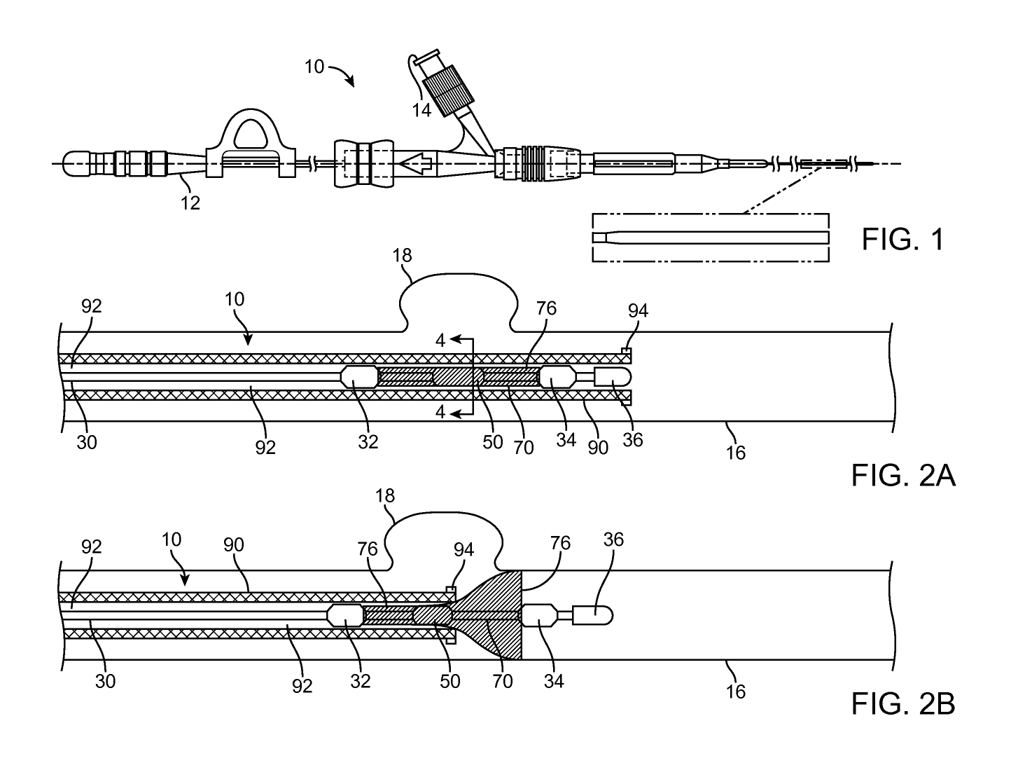

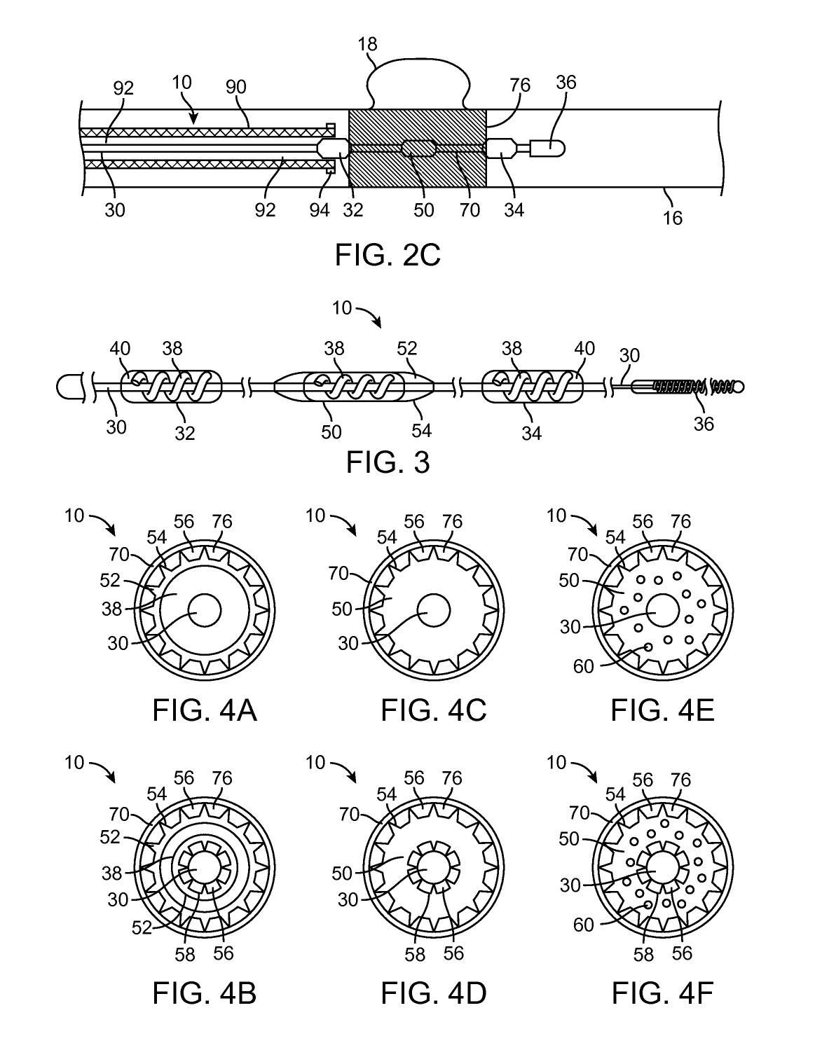

Referring to FIG. 1, the stent delivery system 10 has a handle 12 at its proximal end, which remains outside of the patients and accessible to the operator, when the system 10 is in use. The stent delivery system 10 also has a liquid port 14, which is used to introduce liquid into the system 10 to hydrate a stent 70 mounted therein. FIG. 2A is a schematic view of the stent delivery system 10, having a delivery member 30, a frictional interfacing member 50, a stent 70, and a sheath 90. These parts of the system 10 are located in the distal end of the stent delivery system 10 shown in the inset in FIG. 1.

Still referring to FIG. 2A, the delivery member 30 is a delivery wire 30, which has a proximal bumper 32, a distal bumper 34, and a distal tip 36. Delivery wire 30 may be an elongate member having a proximal end and a distal end. Delivery wire 30 may be made of a conventional guidewire, torqueable cable tube, or a hypotube. In either case, there are numerous materials that can be used for the delivery wire 30 to achieve the desired properties that are commonly associated with medical devices. Some examples can include metals, metal alloys, polymers, metal-polymer composites, and the like, or any other suitable material. For example, delivery wire 30 may include nickel-titanium alloy, stainless steel, a composite of nickel-titanium alloy and stainless steel. In some cases, delivery wire 30 can be made of the same material along its length, or in some embodiments, can include portions or sections made of different materials. In some embodiments, the material used to construct delivery wire 30 is chosen to impart varying flexibility and stiffness characteristics to different portions of delivery wire 30. For example, the proximal region and the distal region of delivery wire 30 may be formed of different materials, for example materials having different moduli of elasticity, resulting in a difference in flexibility. For example, the proximal region can be formed of stainless steel, and the distal region can be formed of a nickel-titanium alloy. However, any suitable material or combination of material may be used for delivery wire 30, as desired.

Delivery wire 30 may further include a distal shapeable or pre-shaped tip 36, which may have an atraumatic distal end to aid in delivery wire 30 advancement. In some cases, distal tip 36 may include a coil placed over a portion of a distal end of the delivery wire 30 or, alternatively, may include a material melted down and placed over a portion of the distal end of delivery wire 30. In some cases, the distal tip 36 may include a radiopaque material to aid in visualization. Although not shown in the Figures, it is contemplated that a distal end of delivery wire 30 may include one or more tapered sections, as desired.

Delivery wire 30 may optionally include one or more bands (not shown) in a distal region of delivery wire 30. Bands may be formed integrally into the delivery wire 30, or they may be separately formed from delivery wire 30 and attached thereto. In some cases, the bands may be disposed on delivery wire 30. The bands may have a diameter greater than the diameter of the surrounding delivery wire 30. Bands may be formed of any suitable material, such as metals, metal alloys, polymers, metal-polymer composites, and the like, or any other suitable material, as well as any radiopaque material, as desired. Alternatively, it is contemplated that the delivery wire 30 may include one or more recesses instead of providing bands, if desired.

As shown in FIG. 3, the proximal and distal bumpers 32, 34 of the delivery wire 30 are made of coils 38 that are attached to the delivery wire 30 and covered with a polymer coating 40. The coils 38 may be wound from platinum wire and soldered to the delivery wire 30. The coils 38 may be radiopaque, in which case they function as markers to facilitate determination of delivery wire position. The coils 38 may have open and/or closed pitch. Alternatively, the coils 38 may be machined, micro-machined, laser cut, or micro-molded from any suitable material. The distal tip 36 is floppy and steerable using pull wires (not shown) to facilitate tracking of the stent delivery system 10 through a vessel 16 to reach a target site 18, such as an aneurysm 18.

The stent delivery system 10 also includes a frictional interfacing member 50 configured to resist longitudinal movement of an overlying stent 70 disposed thereon. The frictional interfacing member 50 is generally a cylindrically shaped elongate body. As shown in FIGS. 3 and 4A, the frictional interfacing member 50 is made of a coil 38 that is attached to a distal region of the delivery wire 30 and covered with a radiopaque or non-radiopaque polymer tubing 52. The coil 38 may be wound with a flat or round wire made from a radiopaque or non-radiopaque metal material. The coil 38 may also be a metal tube or band, or a micro-machined or laser cut thin-walled hypotube or sleeve. The coil 38 material may be platinum, palladium, NiTi or 300 series stainless steel. The coil 38 limits ovalization of the frictional interfacing member 50, which may restrict movement of the delivery wire 30.

The polymer tubing 52 may be made of Pebax® 2533, which is a thermoplastic elastomer made up of block copolymers consisting of a sequence of polyamide and polyether segments. The polymer tubing 52 may also be made from Pebax® 2533 blended with 30% to 80% Tungsten by weight for radiopacity. A low durometer polymer tubing 52 is thermally laminated over the coil 38 to form a tacky outer surface 54. The outer surface 54 contacts to the overlying stent 70 and frictionally resists axial movement of the stent 70 relative to the frictional interfacing member 50 and the delivery wire 30 during deployment and re-sheathing as described below in greater detail. The outer surface 54 of the frictional interfacing member 50, regardless of its composition, has a sufficiently high coefficient of friction to resist axial movement of a compressed stent disposed thereon, without significantly interfering with radial expansion of the compressed stent during delivery.

As shown in cross-section in FIG. 4A, perfusion channels 56 are formed in the outer surface 54 of the frictional interfacing member 50, e.g. by machining, micro-machining, or laser cutting. The perfusion channels 56 fluidly connect the sheath lumen 92 proximal of the frictional interfacing member 50 to the sheath lumen 92 distal of the frictional interfacing member 50. When a liquid, such as hydrating liquid or contrast media, is introduced into the sheath lumen 92 at the proximal end of the sheath 90, the liquid flows through the perfusion channels 56 to hydrate the stent 70 and to hydrate the distal end of the sheath 90.

In an alternative embodiment of the disclosed inventions depicted in FIG. 4B, the coil 38 forming the core of the frictional interfacing member 50 is not soldered to the delivery wire 30. Instead, a polymer tubing 52, like the ones described above, is thermally laminated into the lumen of the coil 38 to form a tacky inner surface 58 and a floating frictional interfacing member 50. In addition, perfusion channels 56 are also formed in the inner surface 54 of the frictional interfacing member 50. Then the floating frictional interfacing member 50 is threaded over the delivery wire 30. Once the frictional interfacing member 50 is on the delivery wire, the tacky inner surface 58 adheres to the delivery wire 30 and frictionally secures the delivery wire 30 to the frictional interfacing member 50. The outer surface 54 of the frictional interfacing member 50 resists axial movement of the stent 70 relative to the frictional interfacing member 50 and the delivery wire 30 during deployment and re-sheathing as described below in greater detail.

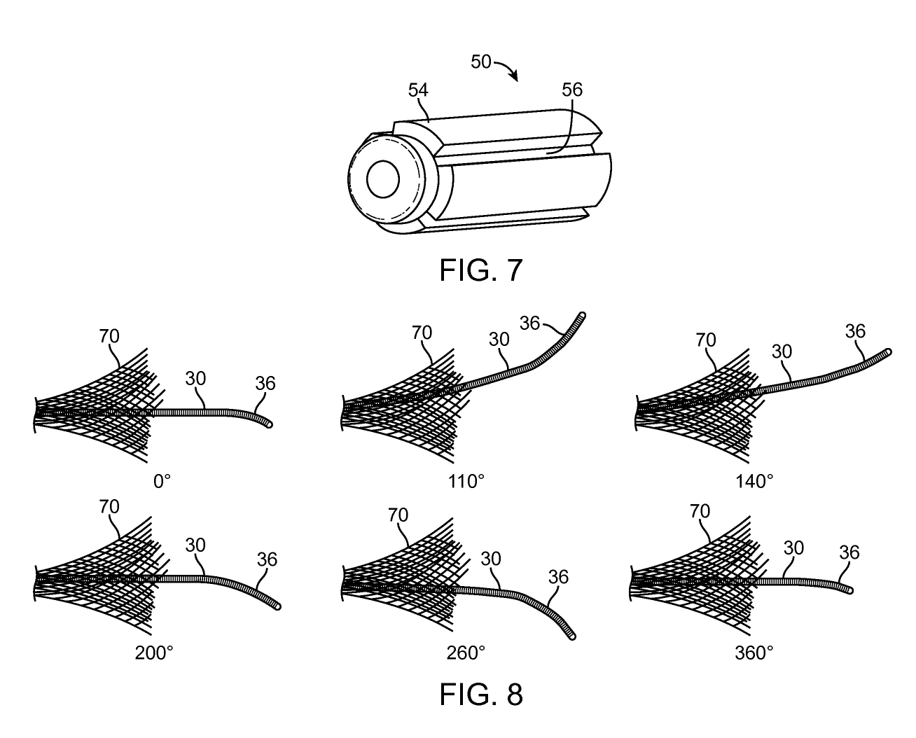

In another alternative embodiment of the disclosed inventions depicted in FIGS. 4C, 4D, and 7, the frictional interfacing member 50 is not reinforced, in that it does not have a coil core. Instead, the frictional interfacing member 50 is extruded or micro-molded as a single piece from a polymer, such as Pebax® 2533 blended with 30% to 80% Tungsten. In the embodiment of the disclosed inventions in FIG. 4C, the frictional interfacing member may be attached to the delivery wire 30 via soldering, lamination, or with a medical grade adhesive. In the embodiment of the disclosed inventions in FIG. 4D, the tacky inner surface 58 of the floating frictional interfacing member 50 adheres to the delivery wire 30 and frictionally secures the delivery wire 30 to the frictional interfacing member 50. The outer surface 54 of the frictional interfacing member 50 resists axial movement of the stent 70 relative to the frictional interfacing member 50 and the delivery wire 30 during deployment and re-sheathing. Like the embodiments of the disclosed inventions in FIGS. 4A and 4B, perfusion channels 56 are formed in the outer surface 54 of the frictional interfacing member 50 in the embodiments in FIGS. 4C and 4D. In addition, perfusion channels 56 are also formed in the inner surface 54 of the frictional interfacing member 50 depicted in FIG. 4D.

The embodiments of the disclosed inventions in FIGS. 4E and 4F are similar to the embodiments in 4C and 4D, respectively. However, the embodiments in FIGS. 4E and 4F have longitudinal perfusion ports 60 formed in the frictional interfacing member 50 via laser drilling. The perfusion ports 60 provide additional liquid paths for liquid to travel past the frictional interfacing member 50.

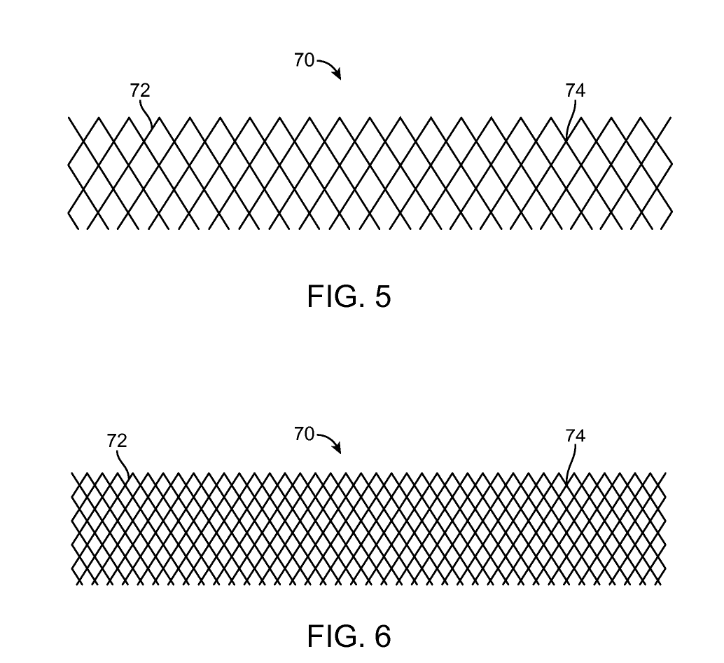

FIG. 5 illustrates a stent 70 for use with the stent delivery system 10. The stent 70 has a closed loop design in that adjacent ring segments 72 are connected at every possible junction 74. However, the stent delivery system 10 may be used with stents having other designs. The stent delivery system 10 may also be used with stents 70 having an overlapping or layered arrangement, as shown in FIG. 6. Overlapping stents 70 may increase the density of coverage or, in other words, decrease the porosity of the cellular configuration or pattern. The increase in the density of coverage may reduce the number of particles that may pass through the stent cells when in use. Such a feature may more effectively divert blood flow away from an aneurysm to help prevent the aneurysm from rupturing.

As illustrated in FIG. 6, the two layers of stent 70 may be longitudinally offset so that the cellular patterns do not completely overlap. For example, the layers may be longitudinally offset by about one-half cell length. However, the layers may be offset by about one-eighth cell length, one-quarter cell length, three-quarter cell length, or any other offset length, as desired. If, however, layers are not offset so that there is complete ring segment 72 overlap due to flow in the vessel or other factors, there may be no or relatively little increase in the density of coverage. Due to the varying degrees of coverage based on the offset or alignment of layers of stent 70, the stent 70 may have a relatively low density of coverage predictability. In some situations, stents having cellular configurations or patterns differing in at least one aspect may increase the predictability of the density of coverage of the assembly. For example, stents having different patterns, mirrored patterns (e.g., left-handedness, right-handedness), different periodicity of patterns, as well as stents of different constructions (e.g., tube, braid) or different materials may be used to help increase the predictability of the density of coverage or cellular porosity.

Further, it is contemplated that the stents 70 may be deployed in an overlapping or layered arrangement or, in other cases, may be interference fit, joined, or otherwise connected to form a multi-layer stent prior to deployment, as desired. In some cases, a single layer stent may be inverted prior to assembly, during deployment, or after deployment to form a multi-layer stent.

For merely illustrative purposes, the foregoing stents 70 have been shown in a flattened view or as a sheet. However, the stents 70 may be rolled into a generally tubular structure, similar to stent 70 shown in FIG. 2A, which may or may not have a generally varied cross-section.

The tubular stent 70 defines a lumen 76 representing the inner volumetric space bounded by the stent 70. The stent 70 is radially expandable from an unexpanded state (FIG. 2A) to an expanded state (FIG. 2C) to allow the stent 70 to expand radially and support the vessel 16. In the illustrative embodiments, the stent 70 is self-expanding. A sheath 90 or other device may be used to radially constrain the stent 70 while being delivered to a target site 18 within the body. When the sheath 90 or other device is retracted proximally from the stent 70, the stent radially expands to a second configuration having a larger diameter, as described in greater detail below.

Further, the foregoing stents 70 may be constructed of any number of various materials commonly associated with medical devices. Some examples can include metals, metal alloys, polymers, metal-polymer composites, as well as any other suitable material. Examples may include stainless steels, cobalt-based alloys, pure titanium and titanium alloys, such as nickel-titanium alloys, gold alloys, platinum, and other shape memory alloys. However, it is contemplated that the foregoing stents 70 may be constructed of any suitable material, as desired. In some cases, different layers of stents 70 may be constructed of different materials, if desired.

Additionally, the foregoing stents 70 may be delivered to a target site 18 by two separate delivery systems 10 to sequentially deliver the stents 70 or, in other cases, by a single multiple stent delivery system. In some cases, the multiple stent delivery system may have the stents 70 mounted thereon in an overlapping arrangement or in a tandem arrangement.

In the illustrative embodiments, the stent 70 may be disposed on a portion of the distal region of delivery wire 30 in a radially constrained first configuration. The stent 70 may be a self-expanding stent. In this example, the stent 70 may be radially constrained by sheath 90 while being delivered to a target site 18 within the body, but when sheath 90 is retracted proximally, the stent 70 may radially expand to a second configuration having a larger diameter.

The stent delivery system 10 includes a retractable sheath 90 disposed over the delivery wire 30 and stent 70. The sheath 90 may take the form of a catheter 90. The sheath 90 may be an elongate tubular member that may have a distal region or end that is disposed over delivery wire 30, having an annular space sufficient in size to receive the radially contracted stent 70 therein. The sheath defines a sheath lumen 92 extending between the proximal and distal ends. The lumen 92 of the catheter 90 is sized to accommodate longitudinal movement of the radially contracted stent 70, the frictional interfacing member 50, and the delivery wire 30. In the illustrative embodiment, movement of sheath 90 in a proximal direction relative to delivery wire 30 may expose the stent 70, allowing expansion of the stent 70.

There are numerous materials that can be used for the sheath 90 to achieve the desired properties that are commonly associated with medical devices. Some examples can include metals, metal alloys, polymers, metal-polymer composites, and the like, or any other suitable material. Examples of suitable metals and metal alloys can include stainless steel, such as 304V, 304L, and 316L stainless steel; nickel-titanium alloy such as a superelastic (i.e., pseudoelastic) or linear elastic nitinol; nickel-chromium alloy; nickel-chromium-iron alloy; cobalt alloy; tungsten or tungsten alloys; tantalum or tantalum alloys, gold or gold alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); or the like; or other suitable metals, or combinations or alloys thereof. Examples of some suitable polymers can include, but are not limited to, polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), polyether-ester, polymer/metal composites, or mixtures, blends or combinations thereof. Sheath 90 can optionally be lined on an inner surface, an outer surface, or both with a lubricious material, if desired.

The catheter 90 may include a braided-shaft construction of stainless steel flat wire that is encapsulated or surrounded by a polymer coating. By way of non-limiting example, HYDROLENE® is a polymer coating that may be used to cover the exterior portion of the delivery catheter 90. Of course, the system 10 is not limited to a particular construction or type of catheter 90 and other constructions known to those skilled in the art may be used for the catheter 90.

The sheath lumen 92 may be advantageously coated with a lubricious coating such as PTFE to reduce frictional forces between the catheter 90 and the stent 70 being moved longitudinally within the lumen 92. The catheter 90 may include one or more optional marker bands 94 formed from a radiopaque material that can be used to identify the location of the distal end of the catheter 90 within the patient's vasculature system or relative to the frictional interfacing member 50 using imaging technology (e.g., fluoroscope imaging).

As shown in FIG. 2A, the stent delivery system 10 may be positioned in the vessel 16 so that stent 70 is positioned adjacent to the target site 18, which in the illustrative example is a weakened region of the vessel 16 or an aneurysm 18. In some cases, the stent 70 may be configured to be deployed across the aneurysm 18 to help divert blood flow in the vessel 16 from entering the aneurysm 18. However, this treatment site is merely illustrative and is not meant to be limiting in any manner. It is contemplated that the delivery system 10 may be used to deliver stents to other target sites, such as stenoses, as desired.

In some cases, the sheath 90 and delivery wire 30 with radially contracted stent 70 may be advanced to the target site, or aneurysm 18, as an assembly. In these cases, the stent delivery system 10 may optionally be inserted into a proximal end of an introducer or other catheter and subsequently advanced to the aneurysm 18. In other cases, the sheath 90 may be advanced to the target site first and then the delivery wire 30 with radially contracted stent 70 may be inserted into a proximal end of sheath 90 and advanced through the sheath lumen 92 to the target site 18.

FIGS. 2A-2C are schematic views of an illustrative procedure for deploying a stent 70 in a vessel 16 using the stent delivery system 10 of FIG. 1. Preliminarily, the stent 70 is mounted around a frictional interfacing member 50 attached to a delivery wire 30. The tacky outer surface 54 of the frictional interfacing member 50 resists axial movement of the stent 70 relative to the frictional interfacing member 50 and the delivery wire 30 during deployment and re-sheathing. Then the stent 70, the frictional interfacing member 50, and the delivery wire 30 are threaded longitudinally into a sheath 90. Next hydrating liquid, such as normal saline, is introduced into the liquid port 14 at the proximal end of the system 10. The hydrating liquid travels through the proximal end of the sheath lumen 92 and the perfusion channels 56 in the outer surface 54 of the frictional interfacing member 50 to the distal end of the sheath lumen 92 to hydrate the stent 70. In some embodiments, such as the ones depicted in FIGS. 4B, 4D, and 4F, the hydrating liquid also travels through the perfusion channels 56 in the inner surface 58 of the floating frictional interfacing member 50. In some embodiments, such as those depicted in FIGS. 4E and 4F, the hydrating liquid also travels through the longitudinal perfusion ports 60 formed in the frictional interfacing member 50.

The distal end of the stent delivery system 10 is then introduced into a vessel 16 containing an aneurysm 18 and advanced to the aneurysm 18. The distal tip 36 of the delivery wire 30 may be steered to track the system 10 through the vessel 16. The embodiments of the disclosed inventions having floating frictional interfacing member 50, e.g. those depicted in FIGS. 4B, 4D, and 4F, the delivery wire 30 may torqued, or rotated about its longitudinal axis, to provide further tracking ability in addition to that provided by steering the distal tip 36. FIGS. 8A to 8F show delivery wire 30 torquing in such an embodiment.

After the stent delivery system 10 has been positioned so that the stent 70 is aligned with aneurysm 18, as shown in FIG. 2A, sheath 90 is partially retracted from the delivery wire 30 exposing a distal portion of stent 70. As illustrated in FIG. 2B, when self-expanding stent 70 is exposed, the stent 70 radially expands to engage a portion of the vessel 16 wall. Optionally, the relative positions of the distal end of the sheath 90 to the frictional interfacing member 50 are monitored while retracting the sheath 90. Radiological visualization of the marker band 94 mounted at the distal end of the sheath 90 and the frictional interfacing member 50 can be used to monitor their relative positions. Such positional monitoring avoids prematurely unsheathing the stent 70 over the frictional interfacing member 50 and releasing the stent 70 from the frictional interfacing member 50.

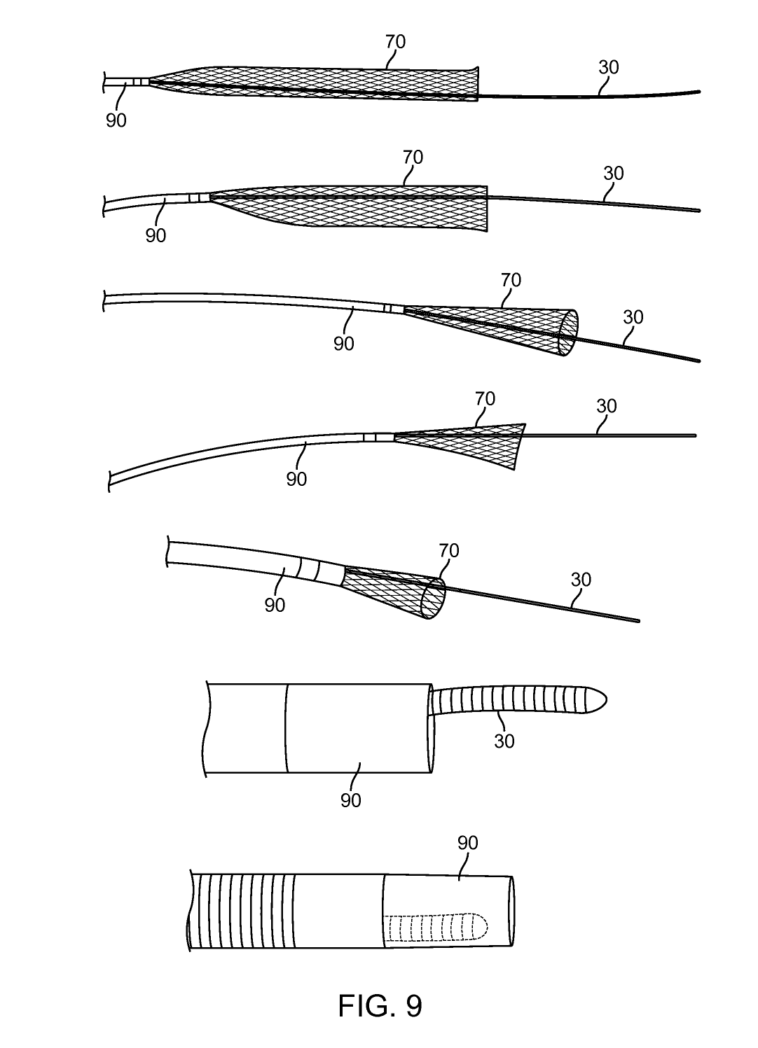

When the stent 70 is partially unsheathed, the position of the stent 70 relative to the aneurysm 18 is determined by radiological visualization. If the position of the partially unsheathed stent 70 is not correct, e.g., misaligned with the aneurysm, the stent 70 is re-sheathed by advancing the sheath 90 distally over the stent 70 or pulling the delivery wire 30 and the stent 70 by way of the frictional interfacing member 50 proximally into the sheath 90. The re-sheathing process is shown in FIGS. 9A to 9G. Using the frictional interfacing member 50 of the above embodiments of the disclosed inventions, an 80% unsheathed stent 70, such as the one shown in FIG. 8A, can be fully re-sheathed. After re-sheathing the stent 70, the sheath 90 and stent 70 contained therein are repositioned based on the previously determined position. The process of partially unsheathing, position determining, re-sheathing, and repositioning is repeated until the position of the partially unsheathed stent 70 relative to the aneurysm 18 is correct.

Next, as illustrated in FIG. 2C, continued retraction of sheath 90 relative to delivery wire 30 to a position proximal of stent 70 completely deploys stent 70. As stent 70 is deployed, the stent 70 fully expands and engages the vessel 16 wall on both sides of aneurysm 18. The stent 70 also expands away from the frictional interfacing member 50.

With stent 70 deployed, delivery wire 30 and frictional interfacing member 50 may be optionally retracted into sheath 90. Then, sheath 90, frictional interfacing member 50, and delivery wire 30 may be withdrawn from the vessel 16 together.

In some embodiments, a degree of MRI compatibility is imparted into catheters. For example, to enhance compatibility with Magnetic Resonance Imaging (MRI) machines, it may be desirable to make the stent delivery system 10 or other portions of the stent delivery system 10 in a manner that would impart a degree of MRI compatibility. For example, delivery wire 30, frictional interfacing member 50, stent 70, sheath 90, or other portions of the stent delivery system 10 may be made of a material that does not substantially distort the image and create substantial artifacts (artifacts are gaps in the image). Certain ferromagnetic materials, for example, may not be suitable because they may create artifacts in an MRI image. Stent delivery systems 10 or portions thereof may also be made from a material that the MRI machine can image. Some materials that exhibit these characteristics include, for example, tungsten, Elgiloy, MP35N, nitinol, and the like, and others. In some embodiments, a sheath and/or coating, for example a lubricious, a hydrophilic, a protective, or other type of material may be applied over portions or all of the stent delivery system 10 or other portions of the system 10.

Although particular embodiments of the disclosed inventions have been shown and described herein, it will be understood by those skilled in the art that they are not intended to limit the present inventions, and it will be obvious to those skilled in the art that various changes and modifications may be made (e.g., the dimensions of various parts) without departing from the scope of the disclosed inventions, which is to be defined only by the following claims and their equivalents. The specification and drawings are, accordingly, to be regarded in an illustrative rather than restrictive sense. The various embodiments of the disclosed inventions shown and described herein are intended to cover alternatives, modifications, and equivalents of the disclosed inventions, which may be included within the scope of the appended claims.

What is claimed is:

1. A stent delivery system, comprising:
a delivery member;
a frictional interfacing member disposed on a distal region of the delivery member, the frictional interfacing member comprising a plurality of perfusion channels;
a self-expanding stent disposed over the respective frictional interfacing member and delivery member in a radially contracted configuration; and
a sheath defining disposed over the respective self-expanding stent, frictional interfacing member, and delivery member,
wherein the frictional interfacing member resists movement of the stent relative to the delivery member while the stent is in its radially contracted configuration, and wherein the perfusion channels permit fluid to flow from an interior region of the sheath proximal of the frictional interfacing member to an interior region of the sheath distal of the frictional interfacing member.

2. The stent delivery system of claim 1, the frictional interfacing member comprising a relatively high friction outer surface that resists rotation of the stent relative to the delivery member while the stent is constrained within the sheath lumen.

3. The stent delivery system of claim 1, the frictional interfacing member comprising a radiopaque core.

4. The stent delivery system of claim 1, further comprising respective proximal and distal bumpers attached to the delivery member and configured to limit respective proximal and distal axial movement of the stent relative to the delivery member while the stent is constrained within the sheath lumen.

5. The stent delivery system of claim 2, wherein at least some of the perfusion channels are formed in the outer surface of the frictional interfacing member.

6. The stent delivery system of claim 1, the frictional interfacing member having an annular body including a high friction inner surface frictionally secured to the delivery member, wherein at least some of the perfusion channels are formed in the inner surface of the frictional interfacing member.

7. The stent delivery system of claim 1, wherein at least some of the perfusion channels comprise ports extending longitudinally through the frictional interfacing member from a proximal facing surface of the frictional interfacing member to a distal facing surface of the frictional interfacing member.

8. The stent delivery system of claim 2, wherein the outer surface of the frictional interfacing member further resists axial movement of the stent relative to the delivery member while the stent is constrained within the sheath.

9. A stent delivery system, comprising:
a delivery member;
an annular frictional interfacing member having a high friction outer surface and a high friction inner surface adhered to the distal region of the delivery member;
a self-expanding stent disposed over the respective frictional interfacing member and delivery member in a radially contracted configuration; and
a sheath disposed over the respective self-expanding stent, frictional interfacing member, and delivery member,
wherein the outer surface of the frictional interfacing member resists axial and rotational movement of the stent relative to the delivery member while the stent is in its radially contracted configuration, and
wherein the frictional interfacing member comprises a plurality of fluid perfusion channels that allow fluid to flow from an interior region of the sheath lumen proximal of the frictional interfacing member to an interior region of the sheath distal of the frictional interfacing member.

10. The stent delivery system of claim 9, further comprising respective proximal and distal bumpers attached to the delivery member and configured to limit respective proximal and distal axial movement of the stent relative to the delivery member while the stent is constrained within the sheath lumen.

11. The stent delivery system of claim 9, wherein at least some of the perfusion channels are formed in the outer surface of the frictional interfacing member.

12. The stent delivery system of claim 9, wherein at least some of the perfusion channels are formed in the inner surface of the frictional interfacing member.

13. The stent delivery system of claim 9, wherein at least some of the perfusion channels comprise ports extending longitudinally through the frictional interfacing member from a proximal facing surface of the frictional interfacing member to a distal facing surface of the frictional interfacing member.

14. The stent delivery system of claim 9, the frictional interfacing member comprising a radiopaque core.

15. A method of delivering a stent to a target site in a blood vessel, comprising:
(a) providing a stent delivery system including a delivery member, a frictional interfacing member disposed on a distal region of the delivery member, a self-expanding stent disposed over the respective frictional interfacing member and delivery member in a radially contracted configuration, and a sheath disposed over the respective self-expanding stent, frictional interfacing member, and delivery member;
(b) introducing liquid into an open proximal end of the sheath, such that the fluid migrates through a plurality of perfusion channels formed in the frictional interfacing member to an interior region of the sheath distal of the frictional interfacing member;
(c) advancing the distal region of sheath into a blood vessel until the stent is positioned proximate a deployment site in the vessel, wherein the frictional interfacing member inhibits rotation of the stent relative to the delivery member during said advancing;
(d) withdrawing the sheath proximally relative to the delivery member to thereby unsheathe a distal portion of the stent, wherein the frictional interfacing member inhibits axial movement of the stent relative to the delivery member during said withdrawing, such that a proximal portion of the stent and the frictional interfacing member remain covered by the sheath;
(e) determining a position of the unsheathed portion of the stent in the vessel; and
(f) if the determined position of the unsheathed portion of the stent is not a desired deployment site in the vessel, advancing the sheath distally relative to the delivery member or withdrawing the delivery member proximally relative to the sheath to thereby re-sheath the distal portion of the stent.

16. The method of claim 15, further comprising
(g) repositioning the distal region of the sheath and re-sheathed stent within the vessel;
(h) repeating acts (d) to (f) until the stent is determined to be at a desired deployment site in the blood vessel;
(i) withdrawing the sheath proximally to unsheathe the entire stent and frictional interfacing member;
(j) allowing the stent to expand radially and disengage from the frictional interfacing member; and
(k) removing the respective sheath, frictional interfacing member, and delivery member from the vessel.

17. The method of claim 15, further comprising monitoring the position of the sheath relative to the frictional interfacing member while withdrawing the sheath proximally over the frictional interfacing member to avoid withdrawing the distal end of the sheath over the frictional interfacing member.

18. The method of claim 17, wherein said monitoring is performed by viewing a radiopaque core of the frictional interfacing member.

19. The method of claim 15, the stent having an axial length, wherein partially unsheathing a distal portion of the stent comprises unsheathing a majority of the axial length of the stent.

20. The method of claim 19, wherein partially unsheathing a distal portion of the stent comprises unsheathing up to about 80% of the axial length of the stent.
